(19) United States
(12) Patent Application Publication
(10) Pub. No.: US 2014/0234433 A1
(43) Pub. Date: Aug. 21, 2014

(54) Topical Composition for Stimulating Epidermis and Dermis Layers of the Skin

(71) Applicants: Nicholas V. Perricone, Meriden, CT (US); Peter T. Pugliese, Bernville, PA (US)

(72) Inventors: Nicholas V. Perricone, Meriden, CT (US); Peter T. Pugliese, Bernville, PA (US)

(21) Appl. No.: 14/181,365

(22) Filed: Feb. 14, 2014

Related U.S. Application Data
(60) Provisional application No. 61/765,542, filed on Feb. 15, 2013.

(51) Int. Cl. A61K 8/98; A61K 8/64; A61Q 19/08; A61K 8/67; A61Q 19/00

(52) U.S. Cl. CPC A61K 8/986; A61K 8/982; A61K 8/671; A61Q 19/00; A61Q 19/08; A61K 8/64
USPC 424/535

(57) ABSTRACT

The invention provides topical compositions including vitamin A, colostrum (or extracts of colostrum with a high IGF content) with or without avian eggshell membrane, that stimulate the epidermis and dermis layers of the skin to treat skin aging.

### FIELD OF THE INVENTION

The present invention relates to topical compositions, comprising vitamin A and insulin like growth factors (“IGF”), and more specifically, topical compositions comprising vitamin A, colostrum (or extracts of colostrum with a high IGF content) with or without avian eggshell membrane that stimulate the epidermis and dermis layers of the skin and treat skin aging. More specifically, the present invention relates to formulations comprising vitamin A, colostrum (or peptide extract of colostrum with a high IGF content), with or without avian eggshell membrane in a dermatologically acceptable carrier, which is useful for improving the appearance of surface spots, brown spots, red areas, wrinkles and texture of skin. A method is provided for preparing these compositions.

### BACKGROUND OF THE INVENTION

Vitamin A plays a role in a variety of functions throughout the body, such as vision, gene transcription, immune function, embryonic development and reproduction, bone metabolism, haematopoiesis, skin and cellular health, and antioxidant activity. Vitamin A is a fat-soluble vitamin which readily enters the skin following topical application. The three major forms of vitamin A are retinol, retinaldehyde, also known as retinal, and retinoic acid. The most commonly used form is the alcohol, retinol. Vitamin A and its analogs are highly effective regulators of cell differentiation, cell proliferation, and apoptosis. Because of these activities, vitamin A and related compounds have been most extensively studied in the contexts of embryonic development and of the cell proliferative process and in relation to skin structures and skin health. Recently, there has been considerable new research interest focused on gaining understanding of the roles that retinoids may have in the development of other metabolic processes such as energy metabolism. The major functions of retinol and skin relate to epidermal proliferation, formation of glycosaminoglycan and connective tissue maintenance. In addition, vitamin A, as retinol, is a well-known antioxidant.

Human skin is constantly directly exposed to the air, solar radiation, environmental pollutants, or other mechanical and chemical insults, which are capable of inducing the generation of free radicals as well as reactive oxygen species (ROS) of our own metabolism. Extrinsic skin damage develops due to several factors: ionizing radiation, severe physical and psychological stress, alcohol intake, poor nutrition, overeating, environmental pollution, and exposure to UV radiation (UVR). It is estimated that among all these environmental factors, UVR contributes up to 80%. UV-induced generation of ROS in the skin develops oxidative stress, when their formation exceeds the antioxidant defiance ability of the target cell. The primary mechanism by which UVR initiates molecular responses in human skin is via photochemical generation of ROS, mainly formation of superoxide anion (O(2) (−) (.)), hydrogen peroxide (H(2)O(2)), hydroxyl radical (OH(.)), and singlet oxygen ((1)O(2)). The only protection of our skin is in its endogenous protection (melanin and enzymatic antioxidants) and antioxidants we consume from the food (vitamin A, C, E, etc.). The most important strategy to reduce the risk of sun UVR damage is to avoid the sun exposure and the use of sunscreens. The next step is the use of exogenous antioxidants orally or by topical application and interventions in preventing oxidative stress and in enhanced DNA repair. Retinol is a very effective antioxidant in the presence of ultraviolet light; in fact, it is believed to be essential for proper working of the skin's defense mechanism against UV radiation.

Vitamin A is metabolized in the skin into retinaldehyde and the biologically active form of vitamin A, retinoic acid. Collectively these compounds are referred to as retinoids. Retinoids are compounds with pleiotropic functions and a relatively selective targeting of certain skin structures. They are vitamins, because retinol (vitamin A) is not synthesized in the body and must be derived from diet, but are also hormones with endocrine activity, because retinol is transformed into molecules that bind to nuclear receptors, exhibit an activity, and are then deactivated. Retinoids exert their effects on target cells by binding and activating nuclear retinoid receptors. Retinoid receptors bind their ligands in form of dimers. Heterodimers can be formed between two different retinoid receptor molecules but also between retinoid X receptors and the vitamin D receptor as well as the triiodothyronin receptor. This indicates complex interactions between retinoids and further hormonal signal transduction molecules. Interaction of retinoid receptors with transcriptional factors activated by other signal transduction mechanisms, e.g. AP-1, may provide dissociation of the retinoid effects. Retinoids can exhibit agonistic activity but also be neutral antagonists and inverse agonists. Topical and oral retinol, tretinoin, isotretinoin, and bexarotene, topical alitretinoin, retinaldehyde, motretinide, adapalene, tazarotene, and systemin acitretin compose the list of launched retinoids.

Psoriasis and related disorders, congenital disorders of keratinization, acne, photoaging and hypovitaminosis A are classical approved indications for retinoid treatment; cutaneous T-cell lymphoma, AIDS-associated Kaposi's sarcoma, acute promyelocytic leukemia and actinic lentigines were recently confirmed as additional indications for retinoid treatment. Retinoids have been successfully used in several other dermatoses, e.g. epithelial precanceroses and tumors, seborrhea, rosacea and acneiform dermatoses, lichen planus, eosinophilic folliculitis, condylomata accuminata, lichen sclerosus and atrophicus.

Retinoids are highly effective antiaging cosmetic compounds when used in the skin. They are able to reverse the damage to the dermis caused by ultraviolet light in the UVA range that is from 315 to 400 nm. One of the main cosmetic applications of retinoids is their use to cause controlled proliferation in the thin epidermis of the aged skin. It is well known that vitamin A is essential to this control of skin health in that it acts both to contain excessive proliferation and to accelerate a lagging or decreased proliferation of the epidermis. Hyper proliferation has been demonstrated from application of certain concentrations of retinol to the skin. In addition, there is a reciprocal relationship between the metabolism of the epidermis and the dermis in that the retinoids, such as retinol, are able to induce collagen synthesis and repair as well as promote regeneration. A major effect of retinol application is in increase in the glycosaminoglycans, particularly in hyaluronic acid.

Vitamin A controls epidermal proliferation, increases the amount of collagen elastin and glycosaminoglycans, acts as an anti-oxidant, helps prevent damage from UVA, makes the skin more supple and soft, and helps to control segment access pigmentation. Because of its many benefits to the skin, there is a desire to create improved compositions comprising vitamin A for use in the treatment of aging and sun damaged skin.

Insulin-Like Growth Factors (IGFs)

Insulin-like growth factors (IGFs) are a group of proteins with high sequence similarity to insulin. IGFs are part of a complex system that cells use to communicate with their physiologic environment. This complex system (often referred to as the IGF “axis”) consists of two cell-surface receptors (IGF1R and IGF2R), two ligands (IGF-1 and IGF-2), a family of six high-affinity IGF-binding proteins (IGFBP-1 to IGFBP-6), as well as associated IGFBP degrading enzymes, referred to collectively as proteases. Insulin-like growth factor 1 (IGF-1) is mainly secreted by the liver as a result of stimulation by growth hormone (GH). IGF-1 is important for both the regulation of normal physiology, as well as a number of pathological states, including cancer. The IGF axis has been shown to play roles in the promotion of cell proliferation and the inhibition of cell death (apoptosis). Insulin-like growth factor 2 (IGF-2) is thought to be a primary growth factor required for early development while IGF-1 expression is required for achieving maximal growth. Factors that are known to cause variation in the levels of GH and IGF-1 in the circulation include an individual's genetic make-up, the time of day, their age, sex, exercise status, stress levels, genetics, nutrition level and body mass index (BMI), disease state, race, estrogen status and xenobiotic intake. IGF-I has an involvement in regulating neural development including neurogenesis, myelination, synaptogenesis, and dendritic branching and neuroprotection after neuronal damage. Increased serum levels of IGF-I in children link to higher IQ. IGF is truly versatile and important biological substance.

The insulin-like growth factor 1 receptor (IGF-1R) is a multifunctional receptor that mediates signals for cell proliferation, differentiation, and survival. Genetic experiments showed that IGF-1R inactivation in skin results in a disrupted epidermis. It has been observed that dermal fibroblasts produce IGF-I, the epidermal basal keratinocytes are IGF-I negative but IGF-I receptor positive, and the keratinocytes of the stratum granulosum produce IGF-I. These observations indicate either that the mitogenesis of the basal keratinocytes is regulated by IGF-I expressed both in the dermis and in the stratum granulosum, or that dermal fibroblasts are responsible for sequestering IGF-I to the basal keratinocytes and that the stratum granulosum-derived IGF-I may be an autocrine regulator of epidermal differentiation. The distribution of IGF-I and its receptor in the hair follicle indicates that IGF-I may be a morphogen, not a mitogen, at those sites, because their proliferating cells, but not their differentiating cells, are IGF-I receptor negative. Further, IGF-I receptor expression by the dermal papilla appears to be switched off during the transition from anagen to catagen, which implies a regulatory role for IGF-I during the hair growth cycle.

The insulin-like growth factor 1 (IGF-1) receptor is critical for epidermal keratinocyte proliferation in vitro, and its expression in normal and psoriatic epidermis suggests that it might regulate keratinocyte proliferation in vivo. In normal skin, IGF-1 receptors are expressed by basal epidermal keratinocytes as well as by basal-like or undifferentiated germinative epithelial cells associated with the follicular outer root sheath, sebaceous glands, and the hair matrix. Hyperplastic epidermis undergoing “regenerative” differentiation (keratin 16+, Ki67+ suprabasal keratinocytes) from psoriasis, chronic skin wounds, and plaques of mycosis fungoides consistently shows increased expression of IGF-1 receptor. In these conditions, the region of expanded IGF-1 receptor expression delimited the epidermal zone of keratinocyte proliferation. This suggests that cell surface IGF-1 receptors are widely expressed by epithelial cells with proliferative potential, that receptor expression can be modulated with differing epidermal growth states, and that these receptors are largely down regulated in highly differentiated epithelial cells.

The appropriate response of human keratinocytes to ultraviolet-B (UVB) is dependent on the activation status of the insulin-like growth factor 1 (IGF-1) receptor. Keratinocytes grown in conditions in which the IGF-1 receptor is inactive inappropriately replicate in the presence of UVB-induced DNA damage. In human skin, epidermal keratinocytes do not express IGF-1, and hence the IGF-1 receptor on keratinocytes is activated by IGF-1 secreted from dermal fibroblasts. It is now known that the IGF-1 produced by human fibroblasts is essential for the appropriate UVB response of keratinocytes. The expression of IGF-1 is silenced in senescent fibroblasts in vitro. Using quantitative reverse transcriptase-PCR and immunohistochemisty, it has been shown that IGF-1 expression is also silenced in geriatric dermis in vivo. The diminished IGF-1 expression in geriatric skin correlates with an inappropriate UVB response in geriatric volunteers. Finally, the appropriate UVB response is restored in geriatric skin in vivo through pretreatment with exogenous IGF-1. These studies provide further evidence for a role of the IGF-1 receptor (IGF-1R) in suppressing UVB-induced carcinogenesis, suggest that fibroblasts have a critical role in maintaining appropriate activation of the keratinocyte IGF-1R, and imply that reduced expression of IGF-1 in geriatric skin could be an important component in the development of aging-related non-melanoma skin cancer. IGF-1, locally produced by skin cells other than keratinocytes, interacts with its receptor, predominantly expressed in basal keratinocytes, to maintain tissue homeostasis.

IGF regulates proliferation of the epidermis; aids in skin repair, is essential for protective response to UVB ultraviolet light, is effective in restoring the protective response to UVB in aging skin, and prevents apoptosis.

Eggshell Membrane

The avian eggshell membrane (ESM) is a bilayer structure formed under the outer hard shell. This membrane may be mechanically separated from the shell and the components extracted to produce a powder that has been found to be effective in wound healing and in the treatment of inflammatory disorders such as rheumatoid and osteoarthritis. The principles of wound healing and anti-inflammatory agents in recent years have received more attention by dermatologists in the study of prevention and correction of aging skin.

The eggshell membrane of chickens is essentially a connective tissue type structure and therefore is composed of fibrous proteins such as collagen type 1, glycosaminoglycans, such as dermatan sulfate and chondroitin sulfate and sulfated glycoproteins including hexosamines, such as glucosamine. In addition, other components identified in eggshell membranes are hyaluronic acid, sialic acid, desmosine and isodesmosine, ovotransferrin, lysyl oxidase, lysozyme, and β-N-acetylglucosaminidase. Various other peptides are also present along with these complex compounds. The discovery of eggshell membrane as a natural source of combined collagen, glucosamine, chondroitin, and hyaluronic acid has prompted the evaluation of this material as a potential treatment for inflammatory disorders of the joints, but the use of eggshell membranes in the treatment of wounds and others disorders of the skin extends back 400 years.

To obtain eggshell membrane, the hard outer shell is first separated from the eggshell membrane in order to create an essentially shell-free membrane. This can be done by various methods either chemical or mechanical but a preferred method is by centrifugation. Separated membrane are submitted to gentle hydrolysis and later dialyzed and purified and then dried produce a high content of protein and moderate quantities of glucosamine (chondroitin sulfate, hyaluronic acid (up to 2%), and collagen, along with additional small molecules including peptides.

Eggshell membrane was traditionally used as a wound covering for burn dressing, as it possesses properties of pain relief, wound protection, and promoted healing. Natural eggshell membrane has antibacterial and antimicrobial activities to resist bacterial invasion and thereby protect the developing. In Japan, Sumo wrestlers use ESM as a natural medicine for injuries.

Wound healing is a four-step sequential event including hemostasis, inflammation, proliferation, and remodeling processes. Essentially damaged tissue has to be repaired; collagen and elastin molecules along with other components of the skin must be made by dermal fibroblast cells. Studies indicate that cell membrane components interact with fibroblasts produce these new structural proteins.

Eggshell membrane might contain almost all extracellular matrix components along with extracellular matrix-regulatory gene products that have been evolutionally conserved in avians. Eggshell membranes have a fibrous network mainly comprised of type V, and X collagen, glucosamine, desmosin, hyaluronic acid. In a low concentration solution, hydrophilic small molecules that are produced by the mild hydrolytic process in a production of eggshell membrane might be assembled in such a manner that large amounts of water surrounding these components mimic younger skin.

Eggshell membrane may also contain insulin-like growth factors (IGFs).

Colostrum

Colostrum is a form of milk produced by the mammary glands of mammals (including humans) in late pregnancy. Most species will generate colostrum just prior to giving birth. Colostrum contains antibodies to protect the newborn against disease, as well as being lower in fat and higher in protein than ordinary milk. Colostrum is known to contain immune cells (as lymphocytes) and many antibodies such as IgA, IgG, and IgM. These are the major components of the adaptive immune system. Inter alia, IgA is absorbed through the intestinal epithelium, travels through the blood, and is secreted onto other Type 1 mucosal surfaces. Other immune components of colostrum include the major components of the innate immune system, such as lactoferrin, lysozyme, lactoperoxidase, complement and proline-rich polypeptides, small messenger peptides that control the functioning of the immune system). Examples include interleukins, tumor necrosis factor, chemokines, and others. Colostrum also contains a number of growth factors, such as insulin-like growth factors I and II, transforming growth factors alpha beta 1 and beta 2, fibroblast growth factors, epidermal growth factor, granulocyte-macrophage-stimulating growth factor, platelet-derived growth factor, vascular endothelial growth factor, and colony-stimulating factor-1. IGF is found in colostrum in significant quantities.

In light of the above, it is desired to provide topical compositions comprising vitamin A and at least one component of eggshell membrane and/or colostrum in a dermatologically acceptable carrier for application to aged skin. More specifically, it is desired to have topical compositions comprising vitamin A, colostrum (or peptide extract of colostrum with a high IGF content), with or without avian eggshell membrane in a dermatologically acceptable carrier for application to skin. It is further desired to provide methods of treating aged or sun damaged skin by applying to aged or sun damaged skin compositions comprising vitamin A and at least one component of eggshell membrane and/or colostrum.

### SUMMARY OF THE INVENTION

The present invention provides topical compositions comprising an effective amount of vitamin A and one or more components of eggshell membrane and/or colostrum in a dermatologically acceptable carrier to treat skin aging, and conditions of aged and sun-damaged skin.

More specifically, the present invention provides topical compositions comprising vitamin A, avian eggshell membrane (or IGF extracted from avian eggshell membrane) and/or colostrum (or IGF extracted from colostrum).

Further, the present invention provides topical compositions comprising vitamin A and insulin like growth factors (“IGF”), and more specifically, topical cream compositions.

Methods for improving the condition of and treating aging and sun-damaged skin comprise applying to skin a composition containing an effective amount of vitamin A, colostrum (or peptide extract of colostrum with a high IGF content), with or without avian eggshell membrane in a dermatologically acceptable carrier.

More specifically, the present invention provides topical compositions and methods of applying compositions comprising approximately 0.1% vitamin A and approximately 0.3% to 0.8% of one or more components (including specifically IGF) of colostrum and/or eggshell membrane in a dermatologically acceptable carrier for treatment of aging and sun-damaged skin and to address surface spots, brown spots, red areas, wrinkles and texture, all visible conditions of aging and sun-damaged skin.

Additional, the invention provides a method for preparing a topical composition comprising a water phase, an oil phase, an aqueous DMAE mixture, vitamin A, one or more components (including specifically IGF rich peptide extracts of colostrum) of eggshell membrane and/or colostrum, and preservatives. The compositions are prepared by mixing the water phase with the oil phase; cooling to about 58° C.; adding preservatives; cooling to about 30° C. then adding vitamin A; cooling to about 28° C. then adding the one or more component of eggshell membrane and/or colostrum; homogenizing; adding the DMAE mixture to the resulting mixture; and overlaying nitrogen.

### BRIEF DESCRIPTION OF THE DRAWINGS

FIG. 1A is a graphical representation of assessment by VISIA analysis of facial surface spots on skin treated with a composition comprising 0.1% retinol and 0.35% extracts of colostrum with a high IGF content.

FIG. 1B is a graphical representation of assessment by VISIA analysis of facial UVB spots on skin treated with a composition comprising 0.1% retinol and 0.35% extracts of colostrum with a high IGF content.

FIG. 1C is a graphical representation of assessment by VISIA analysis of facial brown spots on skin treated with a composition comprising 0.1% retinol and 0.35% extracts of colostrum with a high IGF content.

FIG. 1D is a graphical representation of assessment by VISIA analysis of facial redness on skin treated with a composition comprising 0.1% retinol and 0.35% extracts of colostrum with a high IGF content.

FIG. 1E is a graphical representation of assessment by VISIA analysis of facial fine lines and wrinkles on skin treated with a composition comprising 0.1% retinol and 0.35% extracts of colostrum with a high IGF content.

FIG. 1F is a graphical representation of skin texture score, as assessed by VISIA analysis, of facial skin treated with a composition comprising 0.1% retinol and 0.35% extracts of colostrum with a high IGF content.

FIG. 1G is a graphical representation of assessment by VISIA analysis of pores on facial skin treated with a composition comprising 0.1% retinol and 0.35% extracts of colostrum with a high IGF content.

FIG. 1H is a graphical representation of assessment by VISIA analysis of porphyins—feature count of p. acnes on facial skin treated with a composition comprising 0.1% retinol and 0.35% extracts of colostrum with a high IGF content.

FIG. 2A is a graphical representation of assessment by ballistrometry number of bounces when hammer impacts skin of facial skin treated with compositions a composition comprising 0.1% retinol and 0.35% extracts of colostrum with a high IGF content.

FIG. 2B is a graphical representation of assessment by ballistrometry measurement of amplitude of facial skin treated with compositions a composition comprising 0.1% retinol and 0.35% extracts of colostrum with a high IGF content.

FIG. 2C is a graphical representation of assessment by ballistrometry measurement of skin stiffness of facial skin treated with compositions a composition comprising 0.1% retinol and 0.35% extracts of colostrum with a high IGF content.

FIGS. 3A-3C show spectrofluormetry measurements (295, 340, 375 nm, respectively) of photoaged facial skin treated with a composition comprising 0.1% retinol and 0.35% extracts of colostrum with a high IGF content.

FIG. 4A is a Verhoeff's elastin stain of rat tail treated with a composition comprising 0.1% retinol and 0.35% extracts of colostrum with a high IGF content.

FIG. 4B shows Hematoxylin & Eosin stains of facial skin before and after 2 months of treatment with a composition comprising 0.1% retinol and 0.35% extracts of colostrum with a high IGF content.

FIG. 4C shows Verhoeff's elastin stains of facial skin before and after 2 months of treatment with a composition comprising 0.1% retinol and 0.35% extracts of colostrum with a high IGF content.

FIG. 4D shows Hematoxylin & Eosin stains of facial skin before and after 2 months of treatment with a composition comprising 0.1% retinol and 0.35% extracts of colostrum with a high IGF content.

FIG. 4E shows Verhoeff's elastin stains of facial skin before and after 2 months of treatment with a composition comprising 0.1% retinol and 0.35% extracts of colostrum with a high IGF content.

FIG. 4F shows Hematoxylin & Eosin stains of facial skin before and after 2 months of treatment with a composition comprising 0.1% retinol and 0.35% extracts of colostrum with a high IGF content.

FIG. 4G shows Verhoeff's elastin stains of facial skin before and after 2 months of treatment with a composition comprising 0.1% retinol and 0.35% extracts of colostrum with a high IGF content.

FIG. 4H shows Hematoxylin & Eosin stains of facial skin before and after 2 months of treatment with a composition comprising 0.1% retinol and 0.35% extracts of colostrum with a high IGF content.

FIG. 4I shows Verhoeff's elastin stains of facial skin before and after 2 months of treatment with a composition comprising 0.1% retinol and 0.35% extracts of colostrum with a high IGF content.

FIG. 4J shows Hematoxylin & Eosin stains of facial skin before and after 2 months of treatment with a composition comprising 0.1% retinol and 0.35% extracts of colostrum with a high IGF content.

FIG. 4K shows Verhoeff's elastin stains of facial skin before and after 2 months of treatment with a composition comprising 0.1% retinol and 0.35% extracts of colostrum with a high IGF content.

FIG. 5A is a graphical representation of assessment by VISIA analysis of facial surface spots on skin treated with a composition comprising 0.1% retinol and 0.70% extracts of colostrum with a high IGF content/eggshell membrane.

FIG. 5B is a graphical representation of assessment by VISIA analysis of facial UVB spots on skin treated with a composition comprising 0.1% retinol and 0.70% extracts of colostrum with a high IGF content/eggshell membrane.

FIG. 5C is a graphical representation of assessment by VISIA analysis of facial brown spots on skin treated with a composition comprising 0.1% retinol and 0.70% extracts of colostrum with a high IGF content/eggshell membrane.

FIG. 5D is a graphical representation of assessment by VISIA analysis of facial redness treated on skin with a composition comprising 0.1% retinol and 0.70% extracts of colostrum with a high IGF content/eggshell membrane.

FIG. 5E is a graphical representation of assessment by VISIA analysis of facial fine lines and wrinkles on skin treated with a composition comprising 0.1% retinol and 0.70% extracts of colostrum with a high IGF content/eggshell membrane.

FIG. 5F is a graphical representation of assessment by VISIA analysis of skin texture of facial skin treated with a composition comprising 0.1% retinol and 0.70% extracts of colostrum with a high IGF content/eggshell membrane.

FIG. 5G is a graphical representation of assessment by VISIA analysis of pores of facial skin treated with a composition comprising 0.1% retinol and 0.70% extracts of colostrum with a high IGF content/eggshell membrane.

FIG. 5H is a graphical representation of assessment by VISIA analysis of porphyrins feature count of p. acnes of facial skin treated with a composition comprising 0.1% retinol and 0.70% extracts of colostrum with a high IGF content/eggshell membrane.

FIG. 6A is a graphical representation of assessment by ballistrometry number of bounces when hammer impacts skin of facial skin treated with compositions a composition comprising 0.1% retinol and 0.70% extracts of colostrum with a high IGF content/eggshell membrane.

FIG. 6B is a graphical representation of assessment by ballistrometry measurement of skin stiffness of facial skin treated with compositions a composition comprising 0.1% retinol and 0.70% extracts of colostrum with a high IGF content/eggshell membrane.

FIGS. 7A-7C show spectrofluormetry measurements (295, 340, 375 nm, respectively) of photoaged facial skin treated with a composition comprising 0.1% retinol and 0.70% extracts of colostrum with a high IGF content/eggshell membrane.

### DETAILED DESCRIPTION OF THE INVENTION

Compositions of the present invention comprise vitamin A, colostrum (or extracts of colostrum with a high IGF content) with or without avian eggshell membrane in a dermatologically acceptable carrier. When applied to skin, compositions of the present invention show improvement in surface spots, brown spots, red areas, wrinkles and texture.

In accordance with the invention, the term “surface spots” refers to brown or red spots which include freckles, acne marks or scars, hyperpigmentation and vascular lesions. The term “brown spots” refers to those caused by an excess of melanin on and within the skin, these lesions include freckles, melasma, hyperpigmentation and lentigines. The term “red areas” refers to various skin conditions such as acne, rosacea, inflammation and spider veins that have apparent red structures due to the blood vessels and hemoglobin contained in the papillary dermis. The term “wrinkles” refers to fine lines, furrows, folds and creases in the skin that are the direct result of past sun exposure. Wrinkles are associated with decreased skin elasticity. The term “texture” refers to gradations in the skin's color and tone and surface peaks and valleys that are analyzed to measure smoothness.

In accordance with the invention, the term “skin” refers to the epidermal and dermal layers of skin. The term “skin” when used herein is in the broad sense meaning the skin of the face, body, and neck.

The topical compositions of the present invention comprise from approximately 0.01% to 0.5% by weight of vitamin A, more preferably about 0.05% to about 0.15%, most preferably about 0.1% by weight vitamin A. Preferred embodiments, utilize Vitamin A alcohol supplied by BASF under the trade name Retinol 50 P. Retinol 50 P is a 50% stabilized formulation of retinol (vitamin A) in Polysorbate 20. The product is stabilized with Butylhydroxytoluol (BHT) and Butylhydroxyanisol (BHA).

In addition to vitamin A, preferably retinol, the compositions of the present invention comprise at least one component of colostrum or IGF and/or eggshell membrane present from about 0.05% to about 2.0% by weight, more preferably about 0.1% to about 1.0% by weight, most preferably about 0.30% to about 0.8% by weight.

Colostrum is available from commercial sources as a lyophilized powder and is a component of preferred embodiments of the invention. Colostrum is known to those of skill in the art to contain IGF as well as many other beneficial immune cells and antibodies. In the present invention, the preferred form of colostrum is a peptide extract of colostrum which is rich in IGF.

In other embodiments, other forms IGF of may be used in the invention instead of colostrum or a peptide extract of colostrum which is rich in IGF, including recombinant human IGF-1 expressed in E. coli or NSO cells, recombinant mouse IGF-1 expressed in E. coli, and other sources.

The topical compositions optionally, but preferably, also include avian eggshell membrane. In typical preparations, the eggshell membrane is hydrolyzed eggshell membrane powder.

The compositions of the invention may also contain other adjunct ingredients, typically ranging from about 0.05 to about 10% by weight of the composition. Adjunct ingredients include, but are not limited to one or more of: beta carotene, vitamin D3, lipoic acid; α-hydroxy acids such as glycolic acid or lactic acid; ascorbic acid and its derivatives, especially fatty acid esters of ascorbic acid; or tocotrienols and tocotrienol derivatives and vitamin E compositions enriched with tocotrienols or tocotrienol derivatives. Preferred adjunct agents are beta carotene, tocotrienols, and Seppitonic™ M3 by Seppic, which contains magnesium aspartate, zinc gluconate and copper gluconate.

The inventive compositions and methods can be formulated into a lotion, cream, gel or spray by utilization of different proportions of the ingredients and/or by inclusion of thickening agents such as gums or other forms of hydrophilic colloids. The preferred embodiment is a cream or lotion. Another possible embodiment is a solution that may be spayed onto the skin in a fine mist. The lotions, creams, gel and solution are referred to herein as dermally or dermatologically acceptable carriers, and are formulated using conventional techniques known to those of ordinary skill in the art.

The topical composition of the present invention can contain additional ingredients commonly found in skin care compositions and cosmetics, such as, for example, tinting agents, emollients, skin conditioning agents, emulsifying agents, humectants, preservatives, antioxidants, perfumes, chelating agents, etc., provided that they are physically and chemically compatible with other components of the composition.

Preservatives include, but are not limited to, C₁-C₃ alkyl parabens, sorbic acid and phenoxyenthanol, typically present in an amount ranging from about 0.1% to about 2.0% by weight percent, based on the total composition. A preferred preservative is ISP's Optiphen™ Plus, a liquid preservative formulation featuring a blend of phenoxyethanol, sorbic acid and an emollient base.

Emollients, typically present in amounts ranging from about 0.01% to 5% of the total composition include, but are not limited to, hydrocarbons, fatty esters, fatty alcohols, mineral oils, polyether siloxane copolymers, and mixtures thereof. Preferred emollients are squalane, shae butter and isopropyl palmitate (IPP).

Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, and mixtures thereof.

Emulsifiers, typically present in amounts from about 0.5% to about 15% by weight of the composition, include, but are not limited to, stearic acid, cetyl alcohol, stearyl alcohol, steareth 2, steareth 20, acrylates/C10-30 alkyl acrylate crosspolymers, silicones, dimethylethanolamine (DMAE), phosphatidylcholine (PPC) and mixtures thereof. Preferred emulsifiers are sodium hyaluronate, Promulgen-D® (a mixture of 75% cetostearyl alcohol and 25% ethoxylate cetostearyl alcohol sold by Amerchol Corp.), Arlacel 165 (Glyceryl Stearate and PEG-100 Stearate sold by Croda Inc.) silicone (Dow Corning 200 Fluid, 350 CST), DMAE and Phosphlipon 90 G (phosphatidylcholine with 10% fatty acids sold by Phospholipid GmbH).

Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof. A preferred chelating agent is EDTA-Na2.

Supplemental antioxidants, typically present in an amount ranging from about 0.02% to about 5% by weight of the composition, include, but are not limited to, butylated hydroxy toluene (BHT); vitamin C and/or vitamin C derivatives, such as fatty acid esters of ascorbic acid, particularly ascorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; green tea extracts; mixed polyphenols; and mixtures of any of these. Preferred supplemental antioxidants are BHT, BHA and tocotrienols.

Buffering agents are employed in many compositions. It is preferable for compositions of the present invention to be in an acid media. Preferably, the amount of buffering agent is one that results in compositions having a pH ranging from about 2.5 to about 6.0, more preferably from about 3.0 to about 5.5, most preferably from about 3.8 to about 5.0. Typical buffering agents are chemically and physically stable agents commonly found in cosmetics, and can include compounds that are also adjunct ingredients such as citric acid, malic acid, and glycolic acid buffers. The preferred buffering agent is glycolic acid.

Additional ingredients and methods as disclosed in my U.S. Pat. Nos. 5,376,361; 5,409,693; 5,545,398; 5,554,647; 5,574,063; 5,643,586; 5,709,868; 5,879,690; 6,051,244; 6,191,121; 6,296,861; 6,437,004; 6,979,459; 7,037,512; 7,226,608; 7,438,896; 8,414,869; 8,580,742; 8,609,604; and 8,609,618, which are hereby incorporated by reference, may also be used.

Moreover, due to degradation and discoloration that may result from inclusion of vitamin A, it may be advantageous to add various coloring agents and/or package the inventive compositions in metal, plastic or laminate tubes.

Generally in the practice of methods of the invention, the topical composition comprising vitamin A, colostrum (or extracts of colostrum with a high IGF content) with or without avian eggshell membrane in a dermatologically acceptable carrier is topically applied to the skin areas, such as that of the body and face, at predetermined intervals, it generally being the case that gradual improvement in skin appearance and conditions is noted with each successive application. In preferred embodiments, the inventive compositions are applied to the entire face, avoiding the eye area, as needed. In particularly advantageous methods of the invention, the skin is cleansed with a gentle cleanser, such as Cetaphil® Gentle Skin Cleanser, prior to application of the inventive compositions. In preferred embodiments, the composition is applied for 30 to 60 days.

The major cause of aging skin is manifested by wrinkles and is due to alterations in elastic and collagen structures which produce inelastic and stiff skin. Results of viscoelastic studies employing a ballistometer to measure the effects of the inventive compositions and methods showed a significant increase in the number of rebounds from the rebound hammer, which indicates increased elasticity. The parameter measured as skin stiffness showed significant reduction at 1 and 2 months. Stiffness results from a process of collagen alteration known as cross-linking.

Dermal changes were further assessed by spectrofluorometry scans of the skin which were performed employing a Spex® SkinSkan instrument (a non-invasive in-vivo measurement technique) to analyze skin chemistry. Evaluation of the epidermis by tryptophan (an amino acid) content showed significant increases at 1 and 2 months. There was a significant decrease in pepsin-digestible collagen cross-link which is normally increased with aging skin. The collagenase-digestible collagen cross-link was significantly decreased at 1 and 2 months. These three findings indicate a rapid restorative action of the product on both the epidermis and dermis.

Biopsy tissue specimens sectioned and stained using H & E (hematoxylin and eosin) showed significant increases in both epidermal thickness and the proliferative layer known as the granulosum. Greater definition was also seen in the basal layer indicating changes not only in the collagen structure, but significant increases in the amount of dermal collagen. Specific stain for elastin showed increases in the elastin content of the dermis, but also changes in the collagen structure in that elastotic fibers (clumped, non-functional) were restored to normal appearance.

The combination of these ingredients stimulates the epidermis and dermis layers of the skin resulting in improvement in surface spots, brown spots, red areas, wrinkles and texture of skin.

The following examples further describe and demonstrate embodiments within the scope of the present invention. The examples are given solely for the purpose of illustration and are not to be construed as limitations of the present invention, as many variations thereof are possible without departing from the spirit and scope of the invention.

### EXAMPLES

### Example 1

Oil in water emulsions are prepared by combining the following ingredients using conventional mixing techniques.

| | % w/w |
|---|---|
| Phase 1 | |
| Water | Q.S. to 100.00 |
| EDTA-Na₂ | 0.20 |
| Sodium Hyaluronate (1%) | 0.20 |
| PCP | 5.00 |
| Phase 2 | |
| IPP | 3.00 |
| Promulgen-D | 3.00 |
| Glyceryl stearate/PEG-100 | 4.00 |
| stearate | |
| Cetearyl Alcohol 50/50 | 0.70 |
| Phase 3 | |
| Preservative | 0.50 |
| Seppitonic M-3 | 5.00 |
| Phase 4 | |
| Retinol-50 P | 0.20 |
| Phase 5 | |
| Extracts of colostrum with a | 0.35 |
| high IGF content | |
| or | |
| 50-50 Mixture: Egg Shell | 0.70 |
| Membrane and Extracts of | |
| colostrum with IGF content | |
| Phase 6 | |
| DMAE | 0-0.2 |
| Water | 0-2.00 |

Preparation. Dissolve Phase 1 ingredients and heat to 60° C. Disperse and heat Phase 2 ingredients to 58° C. Add Phase 1 to Phase 2 with lightening mixer with slow agitation. Cool to 58° C. and add Phase 3; cool to 30° C. and add Phase 4; cool to 28° C. and add Phase 5. Homogenize the above slurry and start sweep mixing. Add Phase 6 and mix for another 5 minutes and overlay nitrogen and cover overnight. Re-mix next morning and package.

### Example 2

### Determination of Efficacy of a Night Treatment that Contains 0.1% Retinol (Vitamin a) by Ballistometry, VISIA Photography, Spectrofluorometry, and Global Clinical Evaluations

The objective of this clinical research study was to determine the efficacy of a product that contains 0.1% retinol and 0.35% extracts of colostrum with a high IGF content (HNC 157-66 0.1% Vit-A Cream) by ballistometry, photography, spectrofluorometry, and global conical evaluations.

Fifteen healthy females free of systemic and dermatological disease were followed for two months. The women were between the ages of 35-60 years and exhibited moderate facial lines. They did not use any active topical agents on their face during the study, except cosmetic make-up items. Subjects that had used any form of vitamin A, including Retin A®, retinals, retinols and retinoic acids, or α-hydroxy and β-hydroxy acid products were excluded from participating. Five (5) of the participants were biopsied at the start and end of the study. All subjects used the 0.1% Vit-A Cream as directed. There was no placebo treatment in this study.

Subjects were supplied with Cetaphil® Gentle Skin Cleanser to use when cleansing the face and Cetaphil® Moisturizing Cream to use as needed. The 0.1% Vit-A Cream was to be applied to the face daily. The product was applied to the entire face, but subjects were instructed to avoid the eye area. An instruction sheet was provided to the subjects for test product application.

There were three scheduled test sessions: the first was on Day 1-Baseline), at Month 1 (30 days) and at Month 2 (60 days). Additionally, all study participants were asked to return to the laboratory on Day 7 (Week 1) to review the product regime, which helped to ensure study compliance. Global evaluation of aging characteristics of the face were assessed which included fine lines and wrinkles, skin laxity, discolorations, skin texture, and pigmentation changes.

The face was photographed using a VISIA Photographic System (Canfield Imaging, Inc.) that employed a photographic assessment system to record the digital images. These digital photographic images were made of the full frontal face and side views.

Photographs of visible, UV, erythema (redness), porphyrin (p. acnes bacteria) and brown spots were captured. An extensive analysis of the area of interest on the photographs was performed. Areas of interest included: eyes, forehead, cheeks, vertical lines around the mouth, nasolabial and labiomental folds, and pigmented spots (hypopigmentation, hyperpigmentation, and aging spots).

Ballistometry measurements were made to determine changes in the viscoelastic properties of the skin. The Ballistometry system used was similar to the one described in the article by Maes, et. al, although the software program was more sophisticated. The information gained from analysis of the data was translated into terms of elasticity, stiffness, moisturization and suppleness of the skin.

Fluorescent scans of the skin were made using a Spex® SkinSkan spectrofluorometer. Scans were made to measure tryptophan (Ex. 260-340), Em. 340), pepsin-digestible collagen cross-link (Ex. 260-380, Em. 400) and collagenase digestible collagen cross-link (Ex. 260-420, Em. 440).

Five subjects were selected and 2 mm punch biopsies were made at baseline and after 60 days (Month 2). The outer canthus (lateral eye) was chosen as the biopsy field. The skin was infiltrated with 1.0% Lidocaine prior to the punch biopsy.

Results

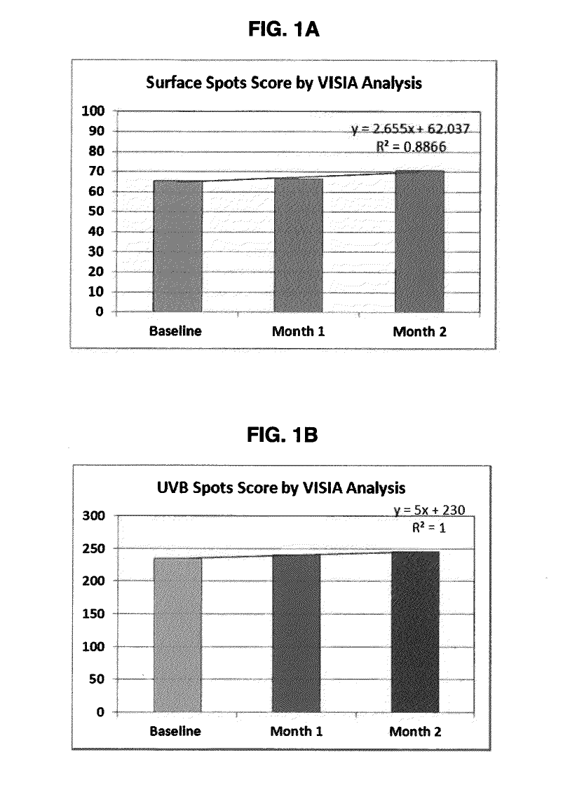

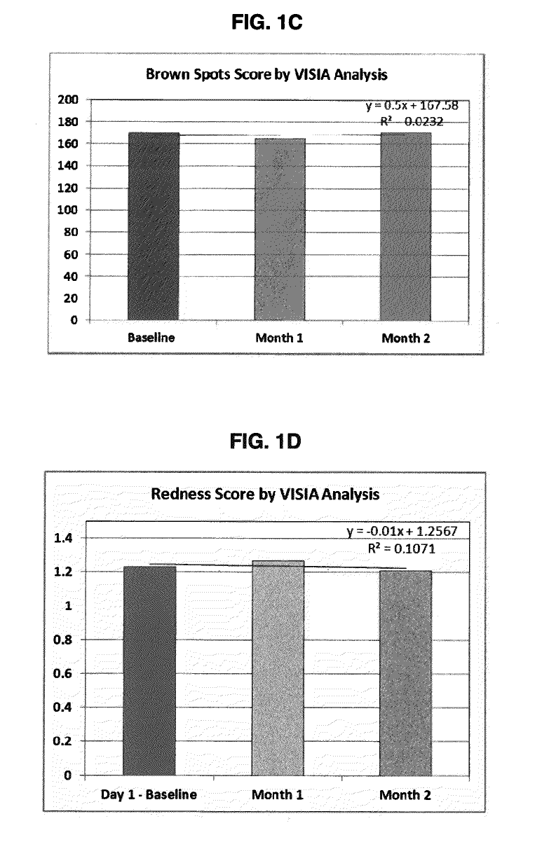

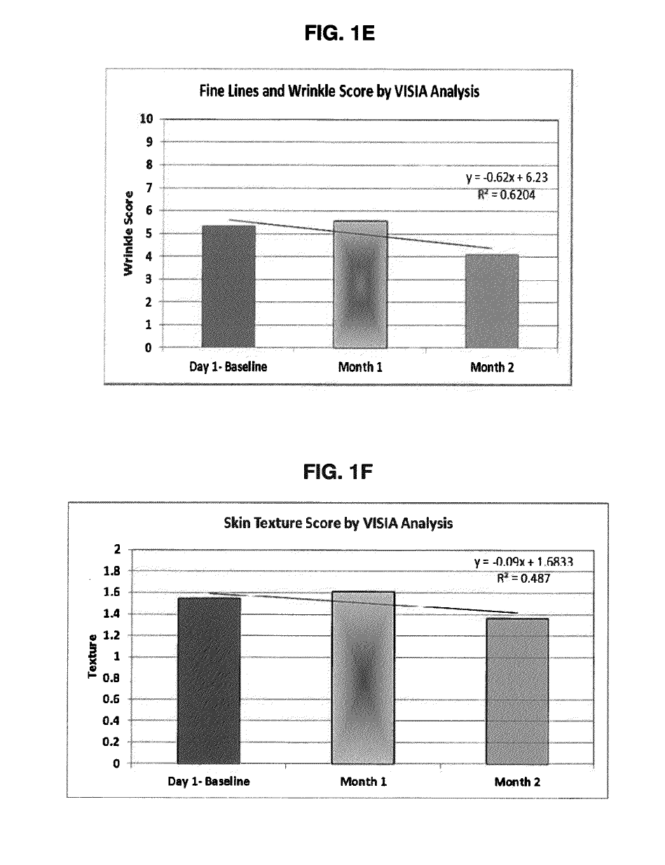

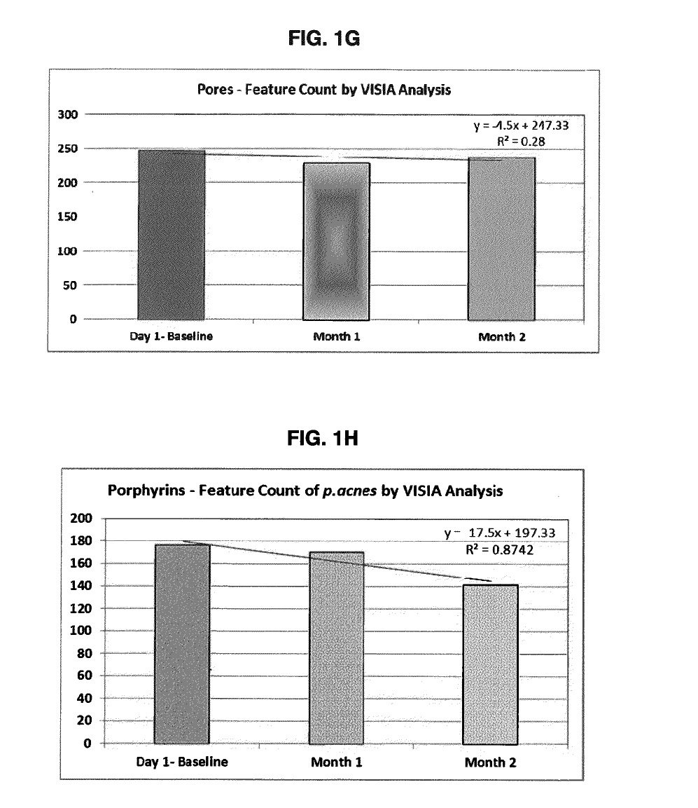

Assessment of VISIA photography is shown in FIGS. 1A-1H. As shown in FIGS. 1A-1C, assessment of surface spots, UV spots and brown spots showed an increase in score. This was expected since as the cells proliferate, pigmented discolorations are brought to the surface where they are sloughed off. Skin redness remained constant as reflected in FIG. 1D. The test product was non-irritating to the skin. As shown in FIG. 1E, there was a 23% reduction in Fine Lines and Wrinkles seen at Month 2. Skin Texture was improved, as reflected in FIG. 1F. As shown in FIG. 1G, pore size was slightly reduced. As shown in FIG. 1H, there was a 20% reduction in the amount Porphyrins at Month 2.

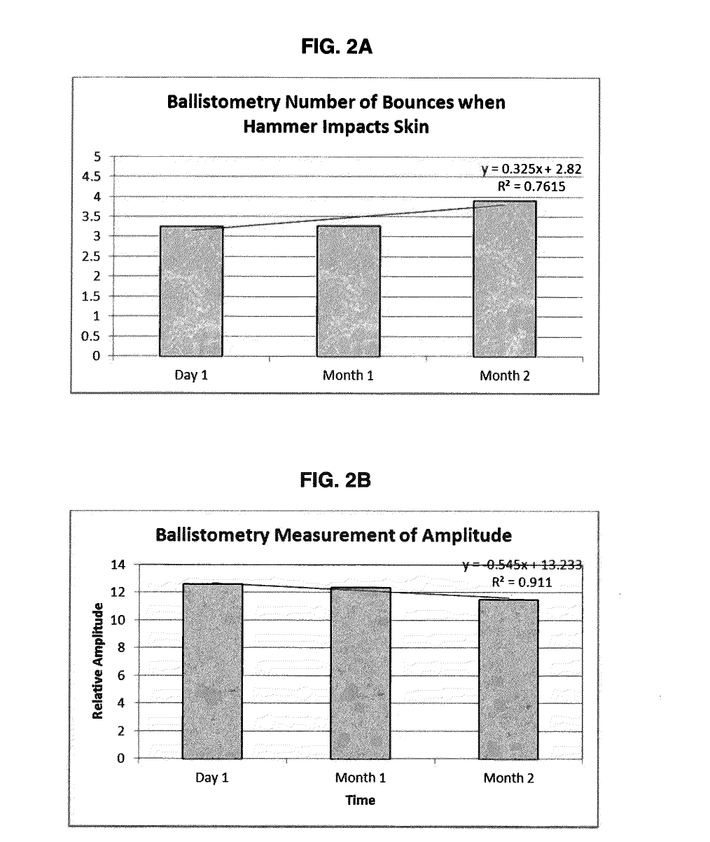

Assessment of Ballistometry measurements is shown in FIGS. 2A-2C. The number of bounces is a good estimate of skin elasticity. As shown in FIG. 2A, there was a 20% increase in the rebound effect.

Cutaneous absorption coefficient or CAC is a dynamic time constant defined for exponential changes in the ballistic arm amplitude. The impact and the rebound energies can be calculated from the CAC value. The CAC is represented as k in the following equation. The CAC increases with age.

Y=Y0e−kt

where Y0 is the initial amplitude of the hammer determined from the initial position of the shaft. Coefficient of Restitution or COR is defined as the ratio of the ballistic arm impact speed to the rebound speed. Skin stiffness is defined in terms of skin “hardness” and is a function of the deformation of the skin on impact. The relative amplitude, shown in FIG. 2B, is a measure of the elasticity as it relates to the rebound energy of the skin.

As shown in FIG. 2C, there was a stiffness increase at month 1, possibly due to the product's moisturization qualities and the hydration effect of the skin. The steady increase at month 1 and 2 are due to changes in the dermal tissue, which indicates firmer skin.

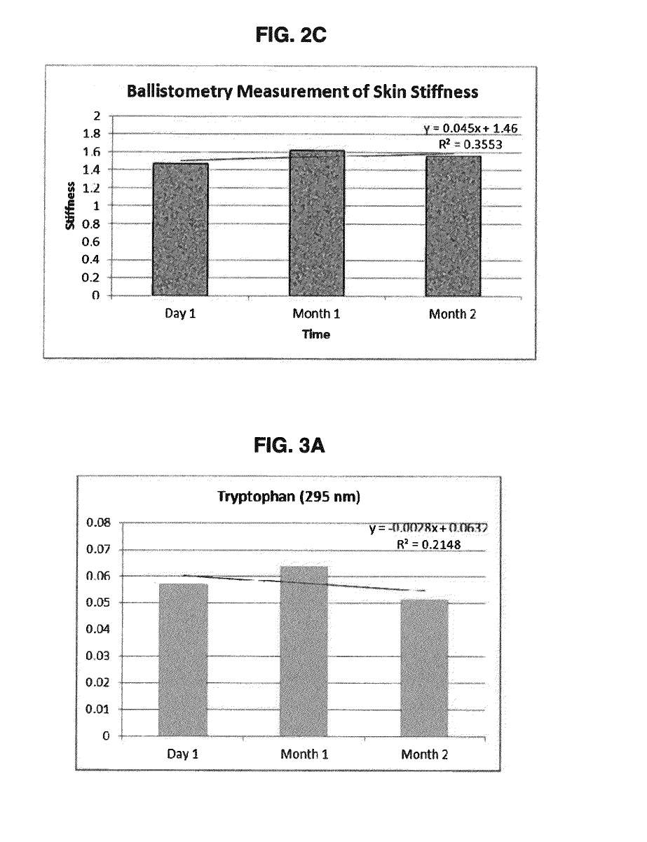

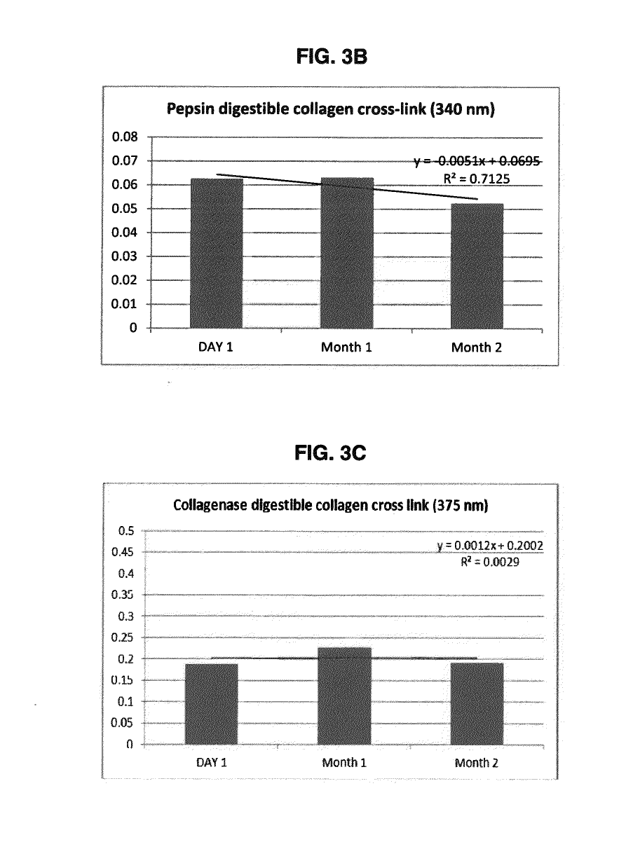

With respect to spectrofluormetry, in photoaged skin the tryptophan and the two distinct collagen bands merge into one broader band, centered at 355 nm. Two new bands (270 nm and 350 nm) appear also. In naturally aged skin, the tryptophan signal decreases, while the pepsin digestible collagen increases. The collagenase-digestible collagen band remains unchanged. Proliferation and production of new collagen in the dermis shows a drop in the intensity of the pepsin-digestible collagen cross link marker. As shown in FIGS. 3A-3C, increased cellular proliferation is seen at 1 month. Aging changes in epidermis are seen as an increase at 295 nm, and in the dermis at 340 nm and 360 nm. Chronological aging produces changes in fluorescence intensity of tryptophan and pdc cross link, but little change in collagenase digestible cross links. The 295 band decreases and the 340 band increases with aging. Chronic UVB exposure induces additional fluorescence excitation bands

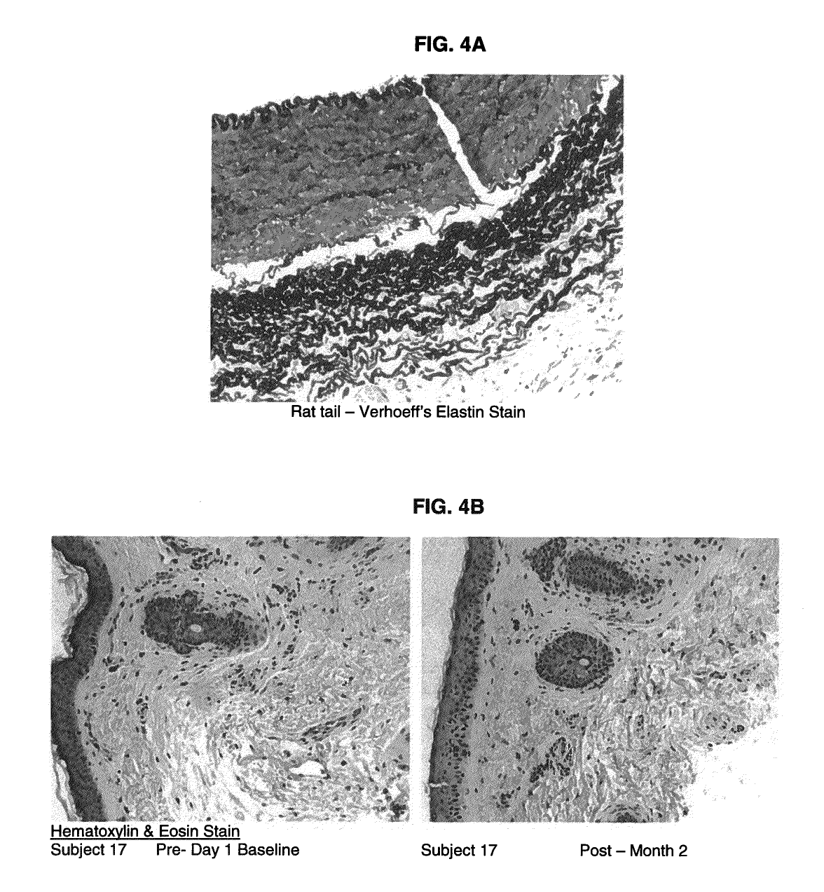

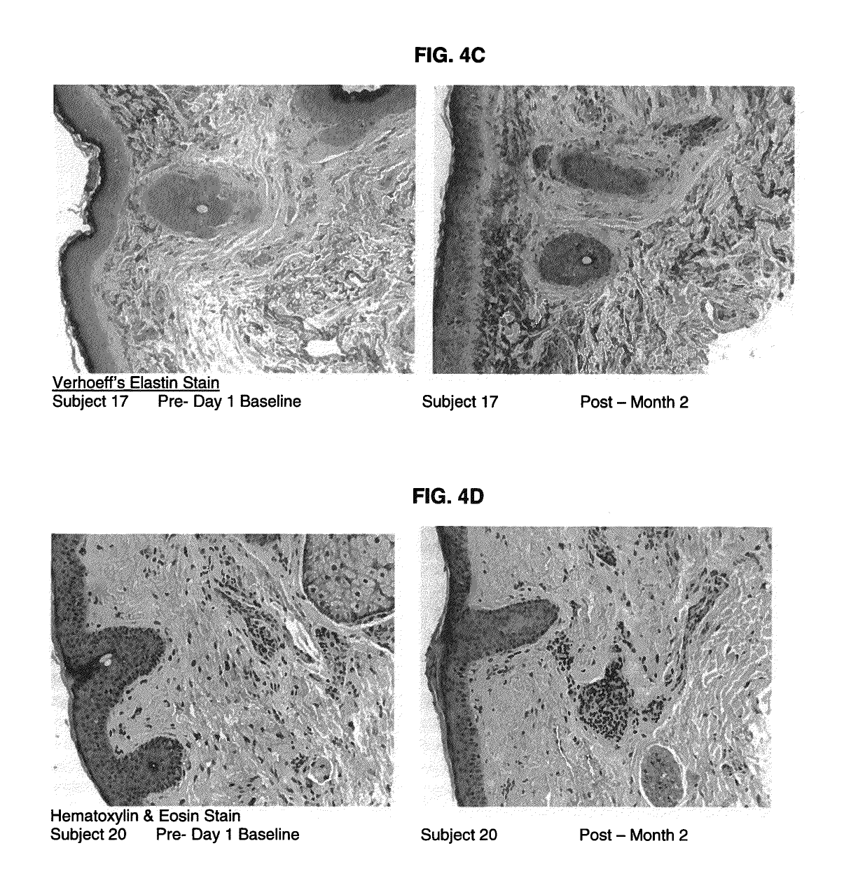

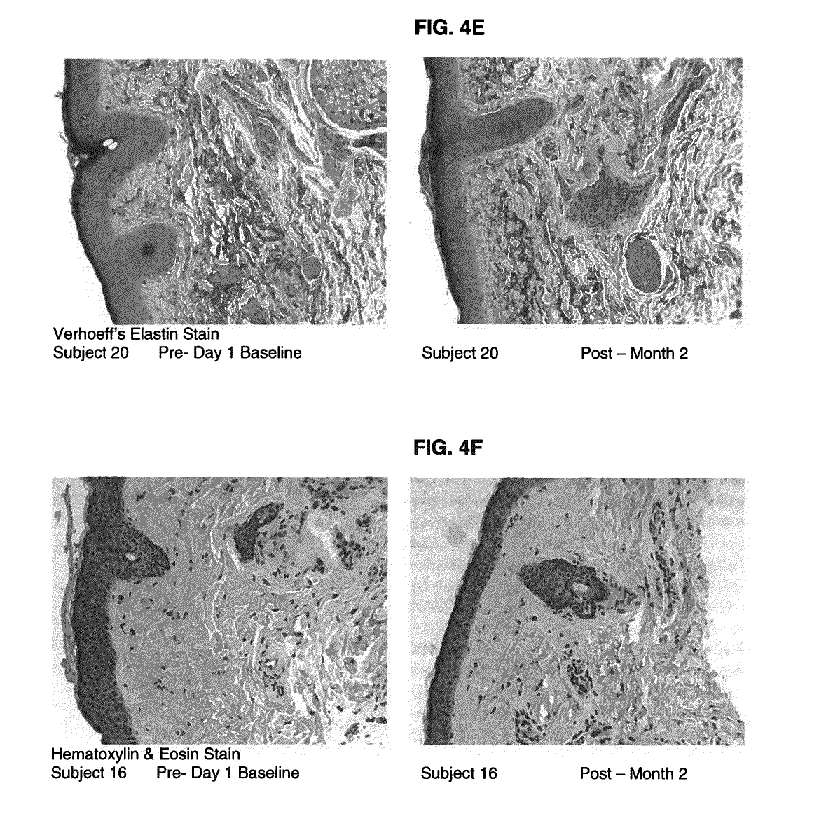

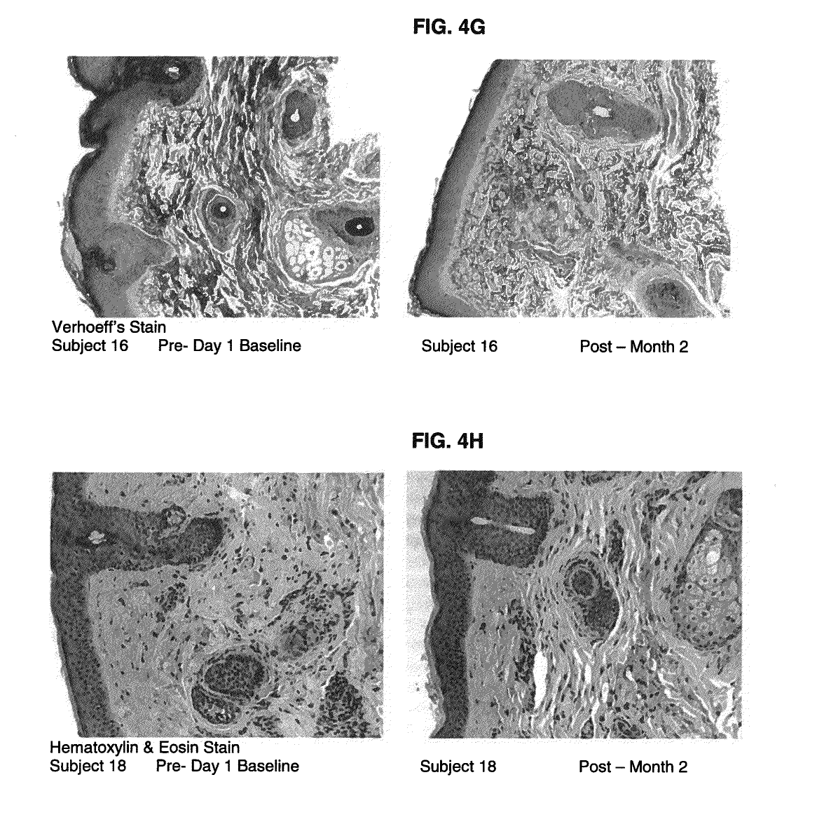

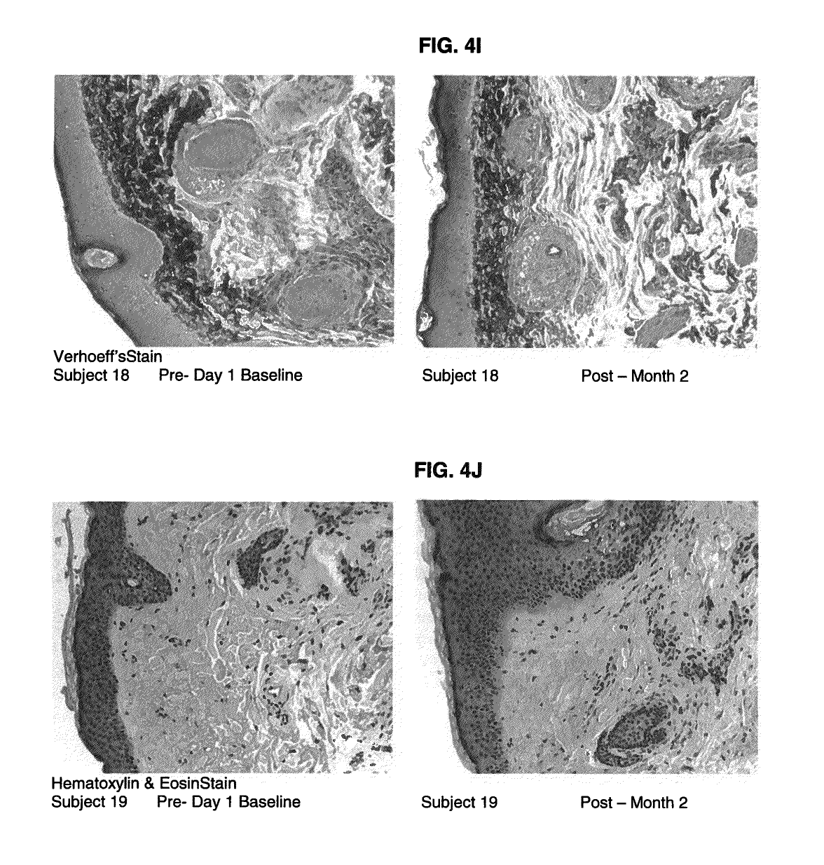

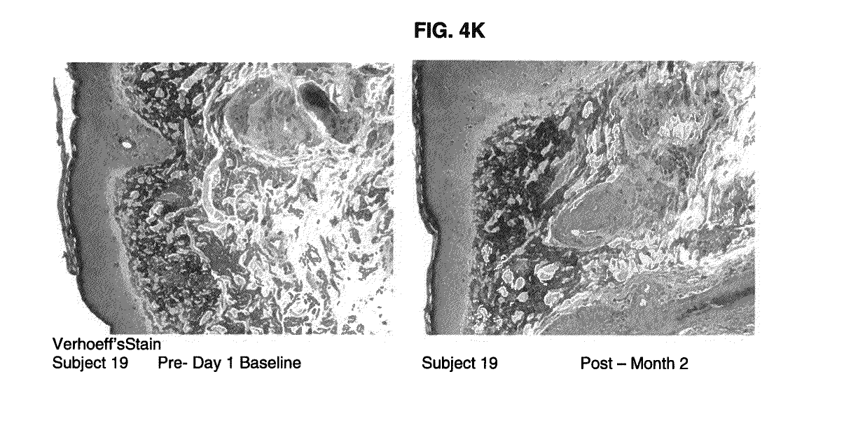

Histological examination of slides is shown in FIGS. 4A-4K. Examination showed that the tissue specimens had a more uniform epidermis. Increased cellularity is seen in the post Month. The tissue appears to be more dense. Three out of five study participants had a marked increase in elastin.

### Example 4

### Determination of the Efficacy of a Night Treatment that Contains 0.1% Retinol (Vitamin A) by Ballistometry, VISIA Photography, Spectrofluorometry, Biopsy and Global Clinical Evaluations

The objective of this clinical research study was to determine the efficacy of a product that contains 0.1% retinol 0.70% extracts of colostrum with a high IGF content/eggshell membrane (HNC 159-31 Cream A) by ballistometry, photography, spectrofluorometry, and global clinical evaluations.

Five healthy females free of systemic and dermatological disease were followed for two months. The women were between the ages of 45-60 years and exhibited moderate facial lines. They did not use any active topical agents on their face during the study, except cosmetic make-up items. Subjects that had used any form of vitamin A, including Retin A®, retinals, retinols and retinoic acids, or α-hydroxy and β-hydroxy acid products were excluded from participating. The participants were biopsied at the start and end of the study. AH subjects used the Cream A as directed. There was no placebo treatment in this study.

Subjects were supplied with Cetaphil® Gentle Skin Cleanser to use when cleansing the face and Cetaphil® Moisturizing Cream to use as needed. The Cream A was to be applied to the face daily. The product was applied to the entire face, but subjects were instructed to avoid the eye area. An instruction sheet was provided to the subjects for test product application.

Test sessions were conducted as above for Example 3. Assessment by VISIA Photography, ballistometry measurements, fluorescent scans and histological evaluation were conducted in the same manner as Example 3.

Results

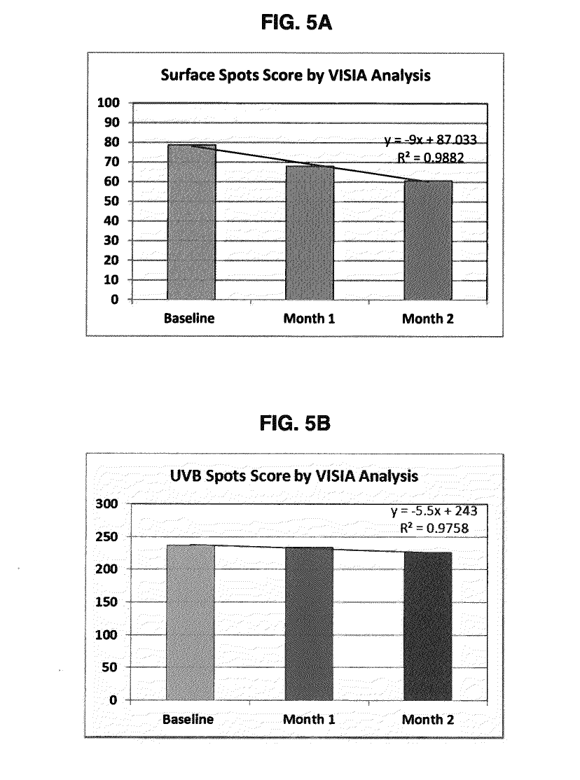

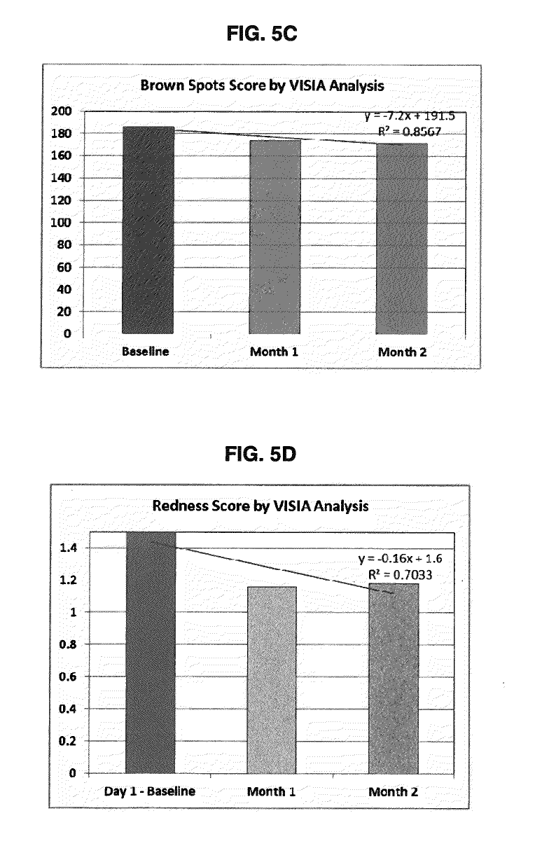

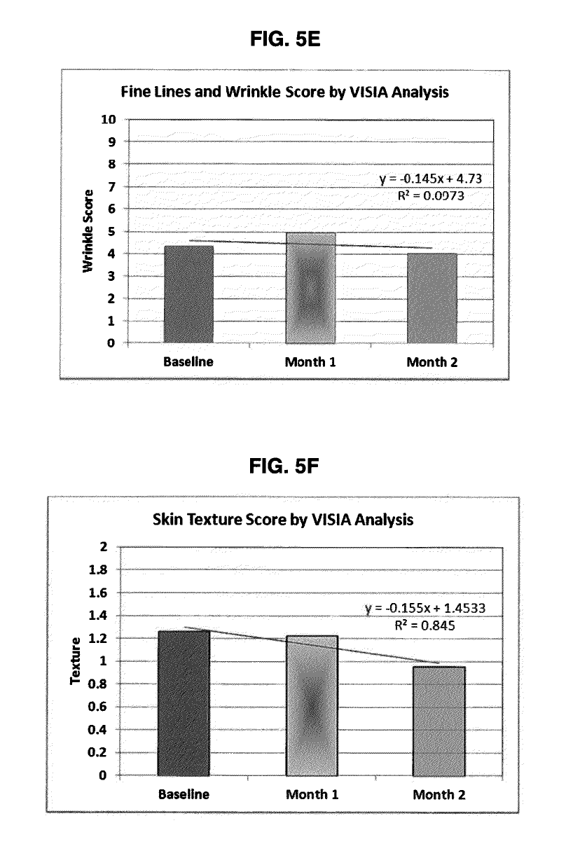

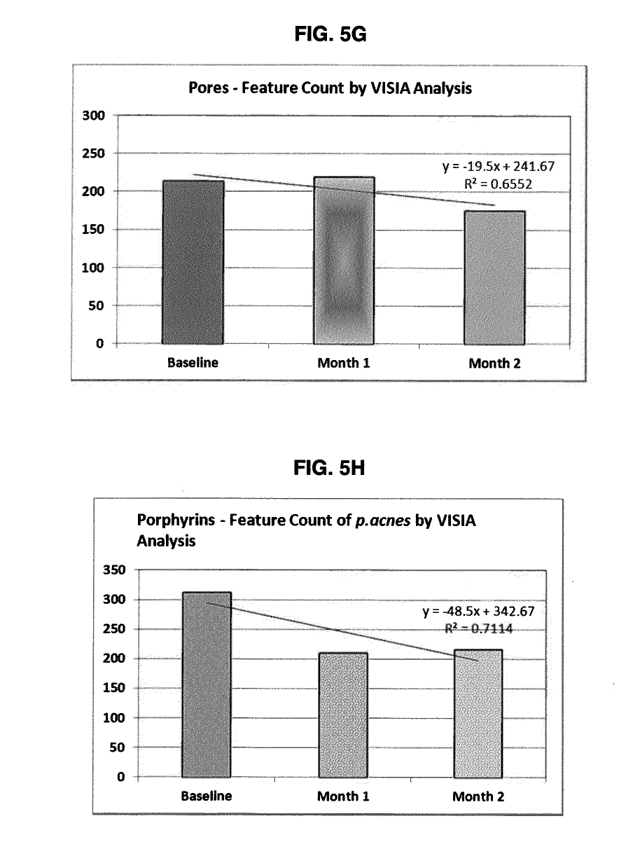

Assessment of VISIA photography is shown in FIGS. 5A-5H. As shown in FIG. 5A, surface spots showed a decrease. The product caused the cells to proliferate which brought the pigmented discolorations to the surface where they were sloughed off. As shown in FIG. 5B-5C, there was no significant change in UV spots or brown spots. As shown in FIG. 5D, skin redness was significantly decreased by greater that 20%. The test product was non-irritating to the skin. As shown in FIG. 5E, there was a reduction in fine lines and wrinkles at Month 2. As shown in FIG. 5F, skin texture was improved by 25% at Month 2. As shown in FIG. 5G, there was reduction in pore size at Month 2. As shown in FIG. 5H, there was greater than a 30% reduction in the amount porphyrins at Month 1 and Month 2. This finding was statistically significant.

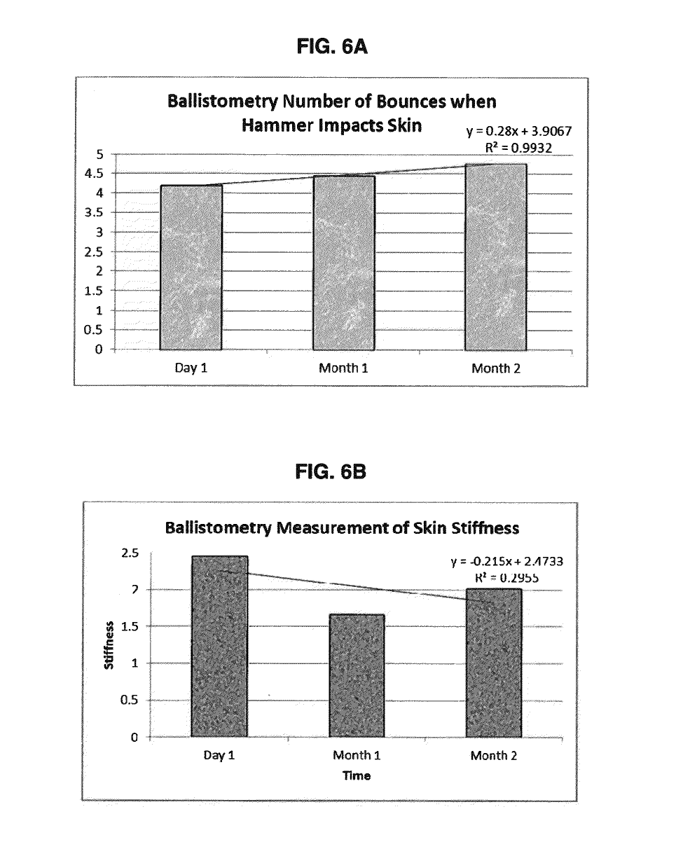

Assessment of ballistometry measurements is shown in FIGS. 6A and 6B. As shown in FIGS. 6A-6B, there was a 20% increase in the rebound effect. The stiffness decrease at Month 1 may be due to the product's moisturization qualities and the hydration effect of the skin. The steady decrease at Month 1 and 2 are due to changes in the dermal tissues which indicates more youthful skin. There was an increase in the rebound effect which means that the skin is more resilient indicating improvement in the extracellular matrix of the skin. There were no significant findings observed with the amplitude or peak height measurements.

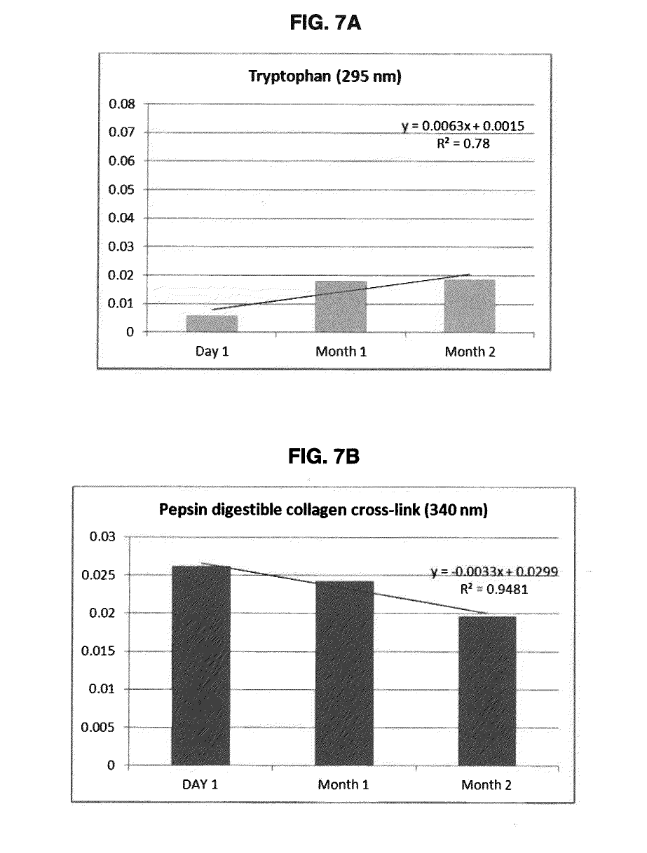

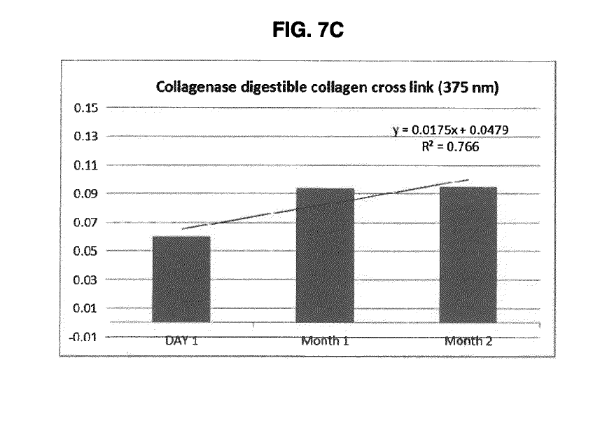

Assessment of spectrofluormetry is shown in FIGS. 7A-7C. As shown in FIGS. 7A-70, the skin appears to be reversing the signs of aging by the epidermal changes seen in the tryptophan band at 295 nm. and in the dermis at 340 nm and 360 nm

### Example 5

The inventive compositions were compared to published studies using 0-0.05% tretinoin (Retin A®). Seven publications were reviewed that described the histological effects of tretinoin at three concentrations when used on facial skin. The study population of several studies involved 533 subjects, 80% of whom were women. The findings from all of the short term studies, that is, 24 months, were the same. Biopsies were taken at the start (Day 1) and at 24 weeks. Biopsy sides were randomized, but both biopsies were taken from adjacent sites on the same side of the face. The findings are summarized as follows.

1. Effect of the Dosage: a) The 0.001% dosage had the same histological characteristics as the inactive base material; b) the 0.01% and 0.05% showed an increased response with an increase in tretinoin concentrations.

2. Specific Histological Findings: a) All studies showed an increase in epidermal thickness due to epidermal hyperplasia; b) all studies showed an increase in the granular layer thickness (1 layer to 2-3 layers); c) all studies showed stratum corneum compactness; d) All studies showed a decrease in melanocytes.

3. Dermal Findings: a) There were no positive findings in the dermis at 24 weeks; b) dermal changes were noted only at 48 weeks of treatment and they were limited; c) continued use up to 4 years, showed a decrease in elastin and perivascular inflammation. In this study the authors concluded that the improvement seen clinically were due to the inflammatory action of the product. (See Ref. 7)

4. Clinical Findings: a) Averaging the studies approximately 80% of the subjects showed erythema, scaling and peeling, and reported stinging.

In comparison, the inventive compositions show no irritation, no redness, no scaling or peeling and no stinging. There was a positive effect in the dermis at 8 weeks showing an increase in collagen and elastin as well as glycosaminoglycans. There was decrease in elastosis, which indicates the ability of the inventive compositions and methods to restore the skin to its normal youthful structure. In conclusion, the inventive compositions and methods are superior to tretinoin 0.5%.

### Example 6

### Consumer Perception Study of a Skin Treatment Product Containing 0.1% Retinol (Vitamin A)

The objective of this study was to evaluate the consumer perception of various attributes of a skin treatment product comprising 0.1% by weight retinal and 0.7% by weight collostrum and IGF in a phosphatidylcholine based carrier in accordance with the present invention after 4 and 8 weeks of product use by a panel of 40 female volunteers between the ages of 35 and 65 years, all of whom were to have moderate skin wing (moderate lines, wrinkles, sun damage, etc.)

Subjects reported to the Clinic on the first day of the study. Subjects were given the skin treatment product (167-35) and daily diary and had digital photos taken of their face. They were instructed to use the skin treatment product by gently smoothing a liberal amount onto clean facial skin each morning avoiding the immediate eye area. During the 8-week test period subjects were instructed not to use any other skin creams/treatments, but could use their regular moisturizer, cleanser and sunscreen. The subjects were also instructed to return to the Clinic after 4 weeks of use to answer a questionnaire and have their product weighed in order to determine rate of use; the return again at 8 weeks to answer a questionnaire, have digital photos taken of their face and have their product weighed in order to determine rate of use.

The majority of subjects (59%-98%) responded positively about all attributes of the product after 4 weeks of use. A total of 98% strongly agreed or agreed that immediately after each application their skin was smoother and softer. Additionally, a total of 95% of the subjects strongly agreed or agreed they saw results without the appearance of redness or irritation. The majority of subjects (64%-100%) responded positively about all attributes of the product after 8 weeks of use. All of the subjects strongly agreed or agreed that immediately after each application, their skin smoother and softer. Additionally, a total 95% of the subjects strongly agreed or agreed they saw results without flaking or peeling.

Subjects noted that an advantage of this embodiment of the invention, a cream formulation, was that it dried fast, had desirable, silky or smooth texture, and had a non-greasy/non-oily feeling when applied to skin. Subjects also touted the composition for its ability to absorb quickly into skin and the ability of makeup to adhere well to the composition.

Insofar as has been determined based upon clinical studies to date, no adverse side effects are encountered with the inventive compositions and methods. The inventive compositions and method are able to restore many of the skin's aging parameters to normal in 30 to 60 days, or less. The compositions and methods are free of any associated irritation frequently seen in retinoic acid products such as redness, scaling skin and increased transepidermal water loss. Overall physiological changes observed in skin are more comprehensive than those employing retinoic acid. It is an advantage of the invention that the inventive compositions are able to be sold over the counter without a prescription.

The above description is for the purpose of teaching the person of ordinary skill in the art how to practice the present invention, and it is not intended to detail all those obvious modifications and variations of it which will become apparent to the skilled worker upon reading the description. It is intended, however, that ail such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A topical composition comprising:
an effective amount of vitamin A;
an effective amount of at least one compound selected from the group consisting of: insulin like growth factors (“IGF”), colostrum, IGF extracted from colostrum, a peptide extract of colostrum which is rich IGF, and avian eggshell membrane; and
a dermatologically acceptable carrier.

2. The topical composition of claim 1, wherein the vitamin A comprises about 0.01% to about 0.5% by weight of the composition.

3. The topical composition of claim 2, wherein the vitamin A comprises about 0.05% to about 0.15% by weight of the composition.

4. The topical composition of claim 3, wherein the vitamin A comprises about 0.1% by weight of the composition.

5. The topical composition of claim 1, wherein the vitamin A is retinol.

6. The topical composition of claim 1, wherein the at least one compound comprises about 0.05% to about 2.0% by weight of the composition.

7. The topical composition of claim 6, wherein the at least one compound comprises about 0.1% to about 1.0% by weight of the composition.

8. The topical composition of claim 7, wherein the at least one compound comprises about 0.30% to about 0.8% by weight of the composition.

9. The topical composition of claim 1, wherein the dermatologically acceptable carrier comprises one or more agents selected from the group consisting of: sodium hyaluronate, phosphatidylcholine, isopropyl palmitate, cetearyl alcohol, glycerol monostearate, and dimethyl ethanolamine.

10. The topical composition of claim 1, wherein the pH of the composition is in the range from about 2.5 to about 6.0.

11. The topical composition of composition of claim 10, wherein the pH of the composition is in the range from about 3.0 to about 5.5.

12. The topical composition of claim 11, wherein the pH of the composition is in the range from about 3.8 to about 5.0.

13. The topical composition of claim 1, wherein the composition is a cream.

14. A topical composition comprising:
about 0.01% to about 0.5% by weight vitamin A;
about 0.05% to about 2.0% by weight avian eggshell membrane or at least one component of eggshell membrane; and
a dermatologically acceptable carrier.

15. The topical composition of claim 14 further comprising about 0.05% to about 2.0% by weight of colostrum or extracts of colostrum rich in IGF content.

16. The topical composition of claim 15, wherein the vitamin A comprises about 0.05% to about 0.15% by weight of the composition.

17. The topical composition of claim 16, wherein the vitamin A comprises about 0.1% by weight of the composition.

18. The topical composition of claim 14, wherein the vitamin A is retinol.

19. The topical composition of claim 15, wherein the eggshell membrane or at least one component of eggshell membrane comprises about 0.35% by weight of the composition.

20. The topical composition of claim 15, wherein colostrum or extracts of colostrum rich in IGF content comprises about 0.35% by weight of the composition.

21. The topical composition of claim 15, wherein the dermatologically acceptable carrier comprises one or more agents selected from the group consisting of: sodium hyaluronate, phosphatidylcholine, isopropyl palmitate, cetearyl alcohol, glycerol monostearate, and dimethyl ethanolamine.

22. The topical composition of claim 15, wherein the pH of the composition is in the range from about 2.5 to about 6.0.

23. The topical composition of composition of claim 22, wherein the pH of the composition is in the range from about 3.0 to about 5.5.

24. The topical composition of claim 23, wherein the pH of the composition is in the range from about 3.8 to about 5.0.

25. The topical composition of claim 15, wherein the composition is a cream.

26. A topical composition comprising:
about 0.1% by weight retinol;
about 0.35% to 0.7% by weight of at least one of peptide extract of colostrum which is rich in insulin like growth factor and optionally eggshell membrane; and
a dermatologically acceptable carrier comprising phosphatidylcholine.

27. The topical composition of claim 26, comprising 0.35% by weight IGF rich peptide extract of colostrum and 0.35% by weight eggshell membrane.

28. A method for preparing a topical composition having a water phase, an oil phase, an aqueous DMAE mixture, vitamin A, one or more of eggshell membrane and colostrum or extracts thereof, and preservative agent, comprising the steps of:
adding the water phase to the oil phase;
mixing the water phase with the oil phase;
cooling to about 58° C.;
adding the preservative agent;
cooling to about 30° C.;
adding the vitamin A;
cooling to about 28° C.;
adding the one more component of eggshell membrane and colostrum;
homogenizing;
adding the DMAE mixture; and
overlaying the resulting mixture with nitrogen.

29. A method for treatment of aging skin, comprising administering to the skin a composition comprising:
an effective amount of vitamin A;
an effective amount of at least one compound selected from the group consisting of: insulin like growth factors (“IGF”), colostrum, IGF extracted from colostrum, a peptide extract of colostrum which is rich IGF, and avian eggshell membrane; and
a dermatologically acceptable carrier.

30. A method for stimulating epidermis and dermis layers of the skin, comprising administering to the skin the composition comprising:
an effective amount of vitamin A;
an effective amount of at least one compound selected from the group consisting of: insulin like growth factors (“IGF”), colostrum, IGF extracted from colostrum, a peptide extract of colostrum which is rich IGF, and avian eggshell membrane; and
a dermatologically acceptable carrier.
